(19) United States
(12) Patent Application Publication
(10) Pub. No.: US 2025/0127971 A1
(43) Pub. Date: Apr. 24, 2025

(54) PERITONEAL DIALYSIS SYSTEM HAVING PERISTALTIC PUMP AND WEIGH SCALE

(71) Applicants: BAXTER INTERNATIONAL INC., Deerfield, IL (US); BAXTER HEALTHCARE SA, Glattpark (Opfikon) (CH)

(72) Inventor: Olof JANSSON, Lund (SE)

(21) Appl. No.: 18/685,133

(22) PCT Filed: Oct. 20, 2022
(86) PCT No.: PCT/US22/78433

§ 371 Date: Feb. 20, 2024

Related U.S. Application Data
(60) Provisional application No. 63/257,761, filed on Oct. 20, 2021.

(51) Int. Cl. A61M 1/28; G16H 40/63

(52) U.S. Cl. CPC A61M 1/281; A61M 1/287; G16H 40/63; A61M 2202/0007; A61M 2202/0014; A61M 2202/04; A61M 2205/103; A61M 2205/106; A61M 2205/127; A61M 2205/3327; A61M 2205/3331; A61M 2205/3365; A61M 2205/3379; A61M 2205/36; A61M 2205/50

(57) ABSTRACT

A peritoneal dialysis system comprises a cycler including a peristaltic pump actuator, and a weigh scale; a disposable set including a peristaltic pump tube operable with the peristaltic pump actuator, a patient line in fluid communication with the peristaltic pump tube, and a heating container in operable communication with the weigh scale; and a control unit configured to cause (i) the peristaltic pump actuator to operate at a rotational speed to cause an amount of fluid to enter the heating container, (ii) weigh the amount of fluid, and (iii) determine at least one of a mass rate or volume rate for the rotational speed.

### PRIORITY CLAIM

The present application claims priority to and the benefit of U.S. Provisional Patent Application No. 63/257,761, filed on Oct. 20, 2021 having the same title as above, the entire contents of which are hereby incorporated by reference and relied upon.

### BACKGROUND

The present disclosure relates generally to medical fluid treatments and in particular to dialysis fluid treatments.

Due to various causes, a person's renal system can fail. Renal failure produces several physiological derangements. It is no longer possible to balance water and minerals or to excrete daily metabolic load. Toxic end products of metabolism, such as, urea, creatinine, uric acid and others, may accumulate in a patient's blood and tissue.

Reduced kidney function and, above all, kidney failure is treated with dialysis. Dialysis removes waste, toxins and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi-or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid, into a patient's peritoneal chamber via a catheter. The dialysis fluid is in contact with the peritoneal membrane in the patient's peritoneal chamber. Waste, toxins and excess water pass from the patient's bloodstream, through the capillaries in the peritoneal membrane, and into the dialysis fluid due to diffusion and osmosis, i.e., an osmotic gradient occurs across the membrane. An osmotic agent in the PD dialysis fluid provides the osmotic gradient. Used or spent dialysis fluid is drained from the patient, removing waste, toxins and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis and continuous flow peritoneal dialysis (“CFPD”). CAPD is a manual dialysis treatment. Here, the patient manually connects an implanted catheter to a drain to allow used or spent dialysis fluid to drain from the peritoneal chamber. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal chamber, wherein the transfer of waste, toxins and excess water takes place. After a dwell period, the patient repeats the manual dialysis procedure, for example, four times per day. Manual peritoneal dialysis requires a significant amount of time and effort from the patient, leaving ample room for improvement.

Automated peritoneal dialysis (“APD”) is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh dialysis fluid and to a fluid drain. APD machines pump fresh dialysis fluid from a dialysis fluid source, through the catheter and into the patient's peritoneal chamber. APD machines also allow for the dialysis fluid to dwell within the chamber and for the transfer of waste, toxins and excess water to take place. The source may include multiple liters of dialysis fluid including several solution bags.

APD machines pump used or spent dialysate from the patient's peritoneal cavity, though the catheter, and to the drain. As with the manual process, several drain, fill and dwell cycles occur during dialysis. A “last fill” may occur at the end of the APD treatment. The last fill fluid may remain in the peritoneal chamber of the patient until the start of the next treatment, or may be manually emptied at some point during the day.

Known APD systems include a machine or cycler that accepts and actuates a pumping cassette having a hard part and a soft part that is deformable for performing pumping and valving operations. Sealing the fluid disposable cassette with a pneumatic path via a gasket to provide actuation has proven to be a potential field issue, which can delay treatment start time and affect user experience. Pneumatic systems are also relatively equipment intensive, often requiring pneumatic storage vessels and structure for pressurizing the storage vessels. Pneumatic systems also produce acoustic noise, which may be a source of customer dissatisfaction.

Known APD systems also produce a good deal of disposable waste. The disposable waste is costly and creates a bioburden.

For each of the above reasons, an improved APD machine is needed.

### SUMMARY

The present disclosure sets forth a streamlined automated peritoneal dialysis (“APD”) cycler and associated system providing a peristaltic pump and disposable set that organizes tubing and performs many functions discussed below. The cycler of the system in one embodiment includes a peristaltic pump actuator that is capable of pumping in two directions. The disposable set includes a peristaltic pump tube that operates with the peristaltic pump actuator. The peristaltic pump tube is connected at both ends to a disposable connector that splits the single tube into multiple inlet/outlet ports. For example, assuming the peristaltic pump actuator and peristaltic pump tube are mounted vertically, an upper end of the peristaltic pump tube may connect to a connector that splits into two inlet/outlet ports, one a heating container port and another a drain line port. A lower end of the peristaltic pump tube may connect to a connector that splits into three or more inlet/outlet ports, one a patient line port and the other two or more ports being dialysis fluid ports.

The disposable set includes a heater line or tube that extends from the heating container port to the heating container or bag. A drain line extends from the drain line port to a house drain, such as a toilet, bathtub or sink. A drain container or bag is not required with the APD cycler and associated system of the present disclosure, which reduces disposable waste and cost. A patient line or tube extends from the patient line port to a patient line connector, which for example connects to a patient's transfer set leading to the patient's indwelling catheter. Dialysis fluid lines or tubes extend from the two or more dialysis fluid ports to dialysis fluid containers or bags, which may hold different dialysis fluid volumes and types, e.g., different glucose or dextrose levels or different formulations, e.g., one dialysis fluid container or bag may include icodextrine for a last patient fill.

The connectors that attach to the end of the peristaltic pump tube may be made, e.g., molded or extruded, of a rigid or non-rigid polymer. In any case, the connectors, fluid lines and fluid containers or bags of the disposable set may be made of one or more plastic, e.g., polyvinylchloride (“PVC”) or a non-PVC material, such as polyethylene (“PE”), polyurethane (“PU”) or polycarbonate (“PC”). The housing of the cycler may be made of any of the above plastics, and/or of metal, e.g., stainless steel, steel and/or aluminum.

The cycler includes or provides a plurality of valves that determine which inlet/outlet port of the connectors is currently open or receiving fluid. The valves in an embodiment are pinch valves that pinch the lines or tubes closed. The pinch valves may be, for example, rotating pinch valves that operate with two or more of the lines or tubes to allow one line or tube to be open, while at the same time the other lines or tubes are occluded. For example, a first rotating pinch valve may be associated with the heater line or tube and the drain line or tube, such that one of the lines or tubes is open, while the other is occluded. A second rotating pinch valve may be associated with first and second dialysis fluid lines or tubes, such that one of the dialysis fluid lines is open, while the other is occluded. A third rotating pinch valve may be associated again with the second dialysis fluid line or tube and the patient line or tube. The combination of the second and third rotating pinch valves allow either of the first and second dialysis fluid lines to be open and the patient line to be occluded or the patient line to be open and the first and second dialysis fluid lines to be occluded. The above valve arrangement allows for fewer valves (e.g., three valves controlling five lines) and thus a more simplified and cost effective cycler.

The heating container or bag (which may or may not be filled with an initial supply of dialysis fluid) is placed on top of a batch heater, for example, a resistive plate heater. The heater may for example include a heater pan that holds the heating container or bag. One or more temperature sensor may be located with the heater pan to sense the temperature of the dialysis fluid within the heating container or bag and to provide feedback for controlling the heater. The heater pan in an embodiment rests on top of one or more load cell, e.g., on top of the cycler. The load cell records the weight of fresh dialysis fluid located within the heating container or bag

The cycler in an embodiment also includes one or more pressure sensor placed along the patient line to read positive and negative pressure of fresh dialysis fluid delivered to and used dialysis fluid removed from the patient. The pressure readings are likewise used as feedback to control the speed of the peristaltic pump actuator. Any of the feedback loops discussed herein, e.g., temperature or pressure, may include a control algorithm such as a proportional, integral and derivative control algorithm. The pressure sensor may be used to ensure that a positive patient pressure limit, for example one to five psig (6.9 to 34.5 kPa, e.g., two psig (13.8 kPa)), is not exceeded during treatment.

The cycler may further include at least one pathogen barrier device, such as an ultraviolet (“UV”) light source placed so as to disinfect the drain line. The pathogen barrier device operating with the drain line is intended to prevent pathogens from migrating, e.g., from the toilet, bathtub or sink, up the drain line and into the peristaltic pumping tube. The cycler may further additionally include a prime sensor, e.g., an optical, capacitance, proximity or magnetic sensor against which the patient line connector is positioned prior to treatment. The prime sensor senses when the patient line has been fully primed.

The cycler includes a control unit having one or more processor, one or more memory and a video controller operating with a user interface to control each of the peristaltic pump actuator, the dialysis fluid valves, the heater and the UV light source, and to receive signals from each of the weigh scale or load cells, temperature sensors, pressure sensor(s) and the prime sensor. The user interface may be provided with a touchscreen and/or electromechanical pushbuttons to allow the user or patient to enter parameters for treatment and a display screen for providing information, such as treatment status information.

The control unit is in one embodiment programmed to calibrate the peristaltic pump. Assuming that the patient is initially full of used dialysis fluid from a prior treatment (but does not have to be for the calibration), the first step in a new treatment is to drain the patient of used dialysis fluid or effluent. The cycler initially drains the patient as much as possible and records the RPM used and the volume of the initial drain by counting peristaltic pump strokes, which is not accurate, but which suffices for the initial drain and for the present time. The cycler also records the associated pressure(s) along the patient line over the counted peristaltic pump strokes. The patient drain may include a prescribed amount of fluid removed, e.g., be a factor such as 1.3 multiplied by the previous fill volume to account for added ultrafiltration (“UF”), or be ended upon the sensing of a characteristic pressure change indicating that the patient is for practical purposes empty.

In a second step, the cycler fills the patient with a prescribed amount of fresh, heated dialysis fluid that is measured accurately by the scale plus an additional amount of fresh dialysis fluid, which may be an amount needed to fill the patient line from the cycler to the patient, and which is an amount sufficient to take a sample for the analysis of the present disclosure.

In a third step, a sample amount (such as the additional amount delivered as mentioned above, e.g., the patient line volume) of dialysis fluid is removed from the patient to the heating container or bag, where it is weighed and measured accurately. The removal is done at a known revolutions per minute (“RPM”) of the peristaltic pump actuator for a known amount of revolutions (or time), including the RPM used for the initial drain. The resulting pressure and weight gain (from which volume may be determined) are measured. The sample may be divided into segments corresponding to different RPM, wherein the RPM may be ramped continuously from zero up to a maximum speed used during the latest drain (or perhaps a latest number of drains). For example, if the maximum drain rate is an RPM corresponding to 250 ml/min, segment sample readings may be taken so as to include RPM expected to yield 100 ml/min, 150 ml/min and 250 ml/min, or other common flowrates. In this manner, the pressures and resulting flowrates for multiple RPM of the peristaltic pump actuator define the pump's behavior during the previous drain and possibly for at least one subsequent drain of the present treatment. The defined behavior forms a transformation function or algorithm that determines flowrate or mass rate (integrated to provide volume or mass) as a function of pressure and RPM (e.g., Q=f(RPM; P)).

In an alternative embodiment discussed in detail herein, a heating container or bag replenishing sequence is used to interrogate the relevant patient drain RPM instead of the sample as discussed above. The peristaltic pump actuator rotates in the relevant patient drain direction for both the sample and the heating container or bag replenishing sequence in one embodiment, making either suitable for determining the transformation functions discussed herein.

In a forth step, the control unit knowing the transformation function and the input parameters of the initial drain (RPM and associated pressures) determines and records an accurate mass rate and/or volume rate for the initial drain. The control unit may then determine (e.g., integrate) the mass or volume of used dialysis fluid removed from the patient at any given time over the initial drain, including the end of the initial drain, by summing individual revolutions multiplied with the stroke volume/mass at each specific revolution based on the pressure when the revolution occurred in time. If the pressure is constant then the stroke volume is constant and the pumped volume will equal the number of revolutions multiplied by the stroke volume, multiplied further by two (e.g., two peristaltic pump rollers enclose an amount of fluid that is transported towards its outlet, wherein one such amount of fluid is released per each half rotation of the peristaltic pump). The previously calculated (not accurate) volume of the initial drain may accordingly be replaced by the more accurate follow-up total weight or volume of the initial drain just described.

The transformation function may be used to provide a better first estimate for the next drain after RPM and pressures for the next drain are recorded. It is known that peristaltic pump tubing wears over the course of treatment, so the resulting flowrate and pressure for a given RPM may be different during a subsequent drain versus a previous drain. It is accordingly contemplated to recalibrate the peristaltic pump in the manner above (by weighing the sample at the RPM and pressures used in the previous drain) after each fill, wherein the recalibration is used for the next drain.

It may be determined that the amount of wear over multiple drains does not significantly affect the performance of the peristaltic pump. If so, it may be possible for the control unit to determine an accurate RPM at which the peristaltic pump actuator is to be operated so as to achieve a prescribed flowrate. For example, suppose the RPM expected to yield 100 ml/min, 150 ml/min and 250 ml/min for certain pressure(s) are instead found via the sampling to instead yield 98 ml/min, 147 ml/min and 245 ml/min, respectively. Here, the control unit may be programmed to notice that the actual flowrates are consistently or on average two percent less than the expected flowrates. The control unit may thereafter increase one or more RPM by two percent in an attempt to generate a prescribed flowrate. The control unit may during a subsequent sample test the increased one or more RPM, where it is weighed and measured accurately to confirm that the prescribed flowrate is met and that the resulting negative pressure is at or within a negative patient pressure limit, e.g., −1.0 psig to −3.0 psig (−6.9 to −20.7 kPa, e.g., −1.3 psig (−9.0 kPa)). If confirmed, the next patient drain can be operated with the peristaltic pump actuator running at the increased one or more RPM, and wherein the negative pressure is monitored to confirm it matches the pressure recorded during the sample.

It is likely that a patient drain of the present system is performed by varying the speed of the peristaltic pump actuator over the course of a drain and in particular slowing down the peristaltic pump actuator at the beginning and/or end of the drain. It is contemplated therefore to weigh samples at different peristaltic pump actuator speeds, which will be used at different stages over the course of a drain. For example, the mass rate or the volume rate (per revolution and/or time) may be reduced by, e.g., by 75%, for a first and/or last 10% of the drain.

The calibration procedure enables the drain amounts to be recorded accurately without having to collect the patient's effluent in a separate drain container for weighing, which would have to be swapped out or loaded with the heating container or bag to use with a single weigh scale. The patient fill amounts are measured by the weigh scale. At the end of treatment, the total amount of used dialysis fluid removed from the patient (e.g., including an initial removal from the previous day's last fill) less the total amount of fresh dialysis fluid delivered to the patient (including present day's last fill amount) yields the overall amount of ultrafiltration (“UF”) removed from the patient over the treatment period.

In light of the disclosure set forth herein, and without limiting the disclosure in any way, in a first aspect of the present disclosure, which may be combined with any other aspect, or portion thereof, a peritoneal dialysis system includes a cycler having a peristaltic pump actuator, and a weigh scale; a disposable set including a peristaltic pump tube operable with the peristaltic pump actuator, and a heating container in operable communication with the weigh scale; and a control unit configured to (i) cause the peristaltic pump actuator to operate at a rotational speed to deliver an amount of fluid to the heating container, (ii) record a weight of the amount of fluid from the weigh scale, and (iii) determine at least one of a mass rate or volume rate for the rotational speed using the recorded weight.

In a second aspect of the present disclosure, which may be combined with any other aspect, or portion thereof, at least one of (i) the fluid is dialysis fluid, purified water or concentrate, or (ii) the control unit is provided with the cycler.

In a third aspect of the present disclosure, which may be combined with any other aspect, or portion thereof, the control unit is further configured to use the at least one of the mass rate or volume rate for the rotational speed to determine a mass or volume of used dialysis fluid removed in a prior drain operated at the rotational speed.

In a fourth aspect of the present disclosure, which may be combined with any other aspect, or portion thereof, the control unit is configured to perform (i) to (iii) after each of a plurality of patient drains.

In a fifth aspect of the present disclosure, which may be combined with any other aspect, or portion thereof, the amount of fluid entering the heating container during (i) is supplied as a sample taken from dialysis fluid provided during a patient fill to the patient.

In a sixth aspect of the present disclosure, which may be combined with any other aspect, or portion thereof, the amount of fluid entering the heating container during (i) is supplied via a heating container replenishing sequence.

In a seventh aspect of the present disclosure, which may be combined with any other aspect, or portion thereof, the cycler further includes a pressure sensor, wherein the control unit records at least one reading from the pressure sensor during (i), the at least one reading associated with the rotational speed.

In an eighth aspect of the present disclosure, which may be combined with any other aspect, or portion thereof, the control unit is further configured to monitor at least one reading from the pressure sensor during a prior drain while actuating the peristaltic pump tube at the rotational speed.

In a ninth aspect of the present disclosure, which may be combined with any other aspect, or portion thereof, the control unit is configured to determine a transformation function that relates flowrate or mass rate as a function of the rotational speed and the at least one reading from the pressure sensor.

In a tenth aspect of the present disclosure, which may be combined with any other aspect, or portion thereof, the transformation function is provided as a lookup table or an algorithm.

In an eleventh aspect of the present disclosure, which may be combined with any other aspect, or portion thereof, the control unit is configured to apply the transformation function to at least one rotational speed used during a prior drain.

In a twelfth aspect of the present disclosure, which may be combined with any other aspect, or portion thereof, the control unit is configured to repeat (i) to (iii) at least one time at different rotational speeds.

In a thirteenth aspect of the present disclosure, which may be combined with any other aspect, or portion thereof, the control unit is configured to repeat (i) to (iii) multiple times starting from a first rotational speed and increasing incrementally to a maximum rotational speed.

In a fourteenth aspect of the present disclosure, which may be combined with any other aspect, or portion thereof, the control unit is configured to use (i) the rotational speed and the at least one different rotational speed occurring during a prior patient drain and (ii) the corresponding mass rate or volume rate for the rotational speed and the at least one different rotational speed to determine the weight or volume of used dialysis fluid removed over the prior patient drain.

In a fifteenth aspect of the present disclosure, which may be combined with any other aspect, or portion thereof, the control unit is configured to use the mass rate or volume rate for the rotational speed and the at least one different rotational speed to determine at least one adjusted rotational speed to use for a subsequent patient drain to achieve a prescribed drain volume.

In a sixteenth aspect of the present disclosure, which may be combined with any other aspect, or portion thereof, a peritoneal dialysis system includes a cycler having a peristaltic pump actuator, a weigh scale, and a pressure sensor, a disposable set including a peristaltic pump tube operable with the peristaltic pump actuator, a patient line in fluid communication with the peristaltic pump tube, wherein the pressure sensor is positioned and arranged to measure pressure in the patient line, and a heating container in operable communication with the weigh scale; and a control unit configured to (i) cause the peristaltic pump actuator to operate at a rotational speed to deliver an amount of fluid to the heating container, (ii) record a weight of the amount of fluid from the weigh scale, (iii) record a pressure of the fluid from the pressure sensor, and (iv) determine a transformation function that relates flowrate or mass rate as a function of the rotational speed and the recorded pressure.

In a seventeenth aspect of the present disclosure, which may be combined with any other aspect, or portion thereof, the control unit is configured to apply the transformation function to at least one rotational speed used during a prior drain.

In an eighteenth aspect of the present disclosure, which may be combined with any other aspect, or portion thereof, a peritoneal dialysis system includes a cycler having a peristaltic pump actuator, and a weigh scale; a disposable set including a peristaltic pump tube operable with the peristaltic pump actuator, and a heating container in operable communication with the weigh scale; and a control unit configured to (i) cause the pump actuator to actuate the pumping portion at a strokes per minute (“SPM”) to deliver an amount of fluid to the heating container, (ii) record a weight of the amount of fluid from the weigh scale, and (iii) determine at least one of a mass rate or volume rate for the SPM using the recorded weight.

In a nineteenth aspect of the present disclosure, which may be combined with any other aspect, or portion thereof, the strokes depend on rotational movement, piston movement, membrane movement or centrifugal movement.

In a twentieth aspect of the present disclosure, which may be combined with any other aspect, or portion thereof, the control unit is configured to repeat (i) to (iii) at least one time at a different SPM.

In a twenty-first aspect of the present disclosure, which may be combined with any other aspect, or portion thereof, the control unit is configured to repeat (i) to (iii) multiple times starting from a first SPM and increasing incrementally to a maximum SPM.

In a twenty-second aspect of the present disclosure, which may be combined with any other aspect, or portion thereof, the control unit is configured to use (i) the SPM and the at least one different SPM occurring during a prior patient drain and (ii) the corresponding mass rate or volume rate for the SPM and the at least one different SPM to determine the weight or volume of used dialysis fluid removed over the prior patient drain.

In a twenty-third aspect of the present disclosure, which may be combined with any other aspect, or portion thereof, the control unit is configured to use the mass rate or volume rate for the SPM and the at least one different SPM to determine at least one adjusted SPM to use for a subsequent patient drain to achieve a prescribed drain volume.

In a twenty-fourth aspect of the present disclosure, which may be combined with any other aspect, or portion thereof, the cycler further includes a pressure sensor, wherein the control unit records at least one reading from the pressure sensor during (i), the at least one reading associated with the SPM.

In a twenty-fifth aspect of the present disclosure, which may be combined with any other aspect, or portion thereof, the control unit is configured to determine a transformation function that relates flowrate or mass rate as a function of the SPM and the at least one reading from the pressure sensor.

In a twenty-sixth aspect of the present disclosure, which may be combined with any other aspect, or portion thereof, the control unit is configured to apply the transformation function to at least one SPM used during a prior drain.

In a twenty-seventh aspect of the present disclosure, which may be combined with any other aspect, or portion thereof, any of the features, functionality and alternatives described in connection with any one or more of FIGS. 1 to 4 may be combined with any of the features, functionality and alternatives described in connection with any other of FIGS. 1 to 4.

It is accordingly an advantage of the present disclosure to provide an APD system having a peristaltic pump.

It is another advantage of the present disclosure to provide an APD system that is portable to ultra-portable.

It is a further advantage of the present disclosure to provide an APD system that eliminates certain sealing issues present in known APD systems.

It is yet a further advantage of the present disclosure to provide an APD pump driven system that eliminates bulky pneumatic equipment associated with certain APD systems.

It is yet another advantage of the present disclosure to provide an APD system that reduces disposable waste.

It is still a further advantage of the present disclosure to provide an APD system having a simplified cycler.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is an elevation view of one embodiment of a system employing a peristaltic pump actuator and weigh scale of the present disclosure.

FIG. 2 is a sectioned perspective view illustrating a peristaltic pumping tube section of one embodiment of the disposable set of the present disclosure in more detail.

FIG. 3 is a process flow diagram illustrating one possible method for operating the peritoneal dialysis system of the present disclosure.

FIG. 4 is an elevation view of an alternative embodiment of a system employing a peristaltic pump actuator and weigh scale of the present disclosure.

### DETAILED DESCRIPTION

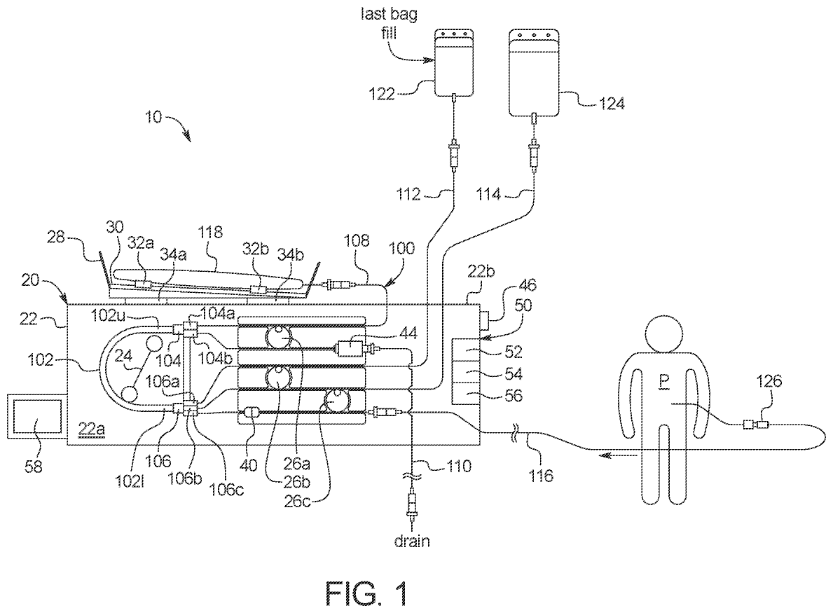

Referring now to the drawings and in particular to FIG. 1, an embodiment of system 10 includes an automated peritoneal dialysis (“APD”) cycler 20 having a housing 22, which uses peristaltic pumping in the illustrated embodiment, and which operates a disposable set 100. All flexible tubing portions of disposable set 100 may be made of one or more plastic, e.g., polyvinylchloride (“PVC”) or a non-PVC material, such as polyethylene (“PE”), polyurethane (“PU”) or polycarbonate (“PC”). Housing 22 of cycler 20 may be made of any of the above plastics, and/or of metal, e.g., stainless steel, steel and/or aluminum. Disposable set 100 organizes tubing and performs many functions discussed below.

Housing 22 includes a front actuation surface 22a, which provides a peristaltic pump actuator 24, which is capable of rotating and pumping in two directions. Peristaltic pump actuator 24 is capable of running at different speeds, thereby creating different flowrates. In an embodiment, drive motor electrical current level sets the speed of peristaltic pump actuator 24. FIG. 1 also illustrates that cycler 20 includes or provides a plurality of valves 26a to 26c, which selectively control the fluid line(s) that is/are presently able to deliver fresh or used dialysis fluid. Valves 26a to 26c in the illustrated embodiment are electrically actuated pinch valves that pinch the lines or tubes of disposable set 100 closed. Pinch valves 26a to 26c may be, for example, rotating pinch valves that operate with two or more of the lines or tubes of disposable set 100 to allow one line or tube to be open, while at the same time the other lines or tubes are occluded. In FIG. 1, rotating pinch valves 26a to 26c each operate with two fluid lines or tubes, wherein each valve simultaneously closes one line and opens another.

FIG. 1 illustrates that cycler 20 includes a heater pan 28 located on a top surface 22b of housing 22. A batch heater 30, for example, a resistive plate heater, is provided to heat heater pan 28 and the dialysis fluid held by the heater pan. One or more temperature sensor 32a, 32b is located with heater pan 28 to sense the temperature of the dialysis fluid and to provide feedback for controlling the heater to controllably heat the dialysis fluid to, e.g., body temperature or 37° C.

Heater pan 28 and heater 30 in the illustrated embodiment rest on top of one or more load cell 34a, 34b forming a weigh scale, which is located on top surface 22b of housing 22 of cycler 20. Load cells 34a, 34b record the weight of fresh dialysis fluid supported by heater pan 28. Load cells 34a, 34b read the weight of the fresh dialysis fluid divided by the number of load cells. If two load cells 34a, 34b are provided, then each load cell is expected to read half the weight of the fresh dialysis fluid. Any suitable number of load cells, e.g., two, four, or more may be provided, wherein the load cells are positioned so as to evenly support heater pan 28.

FIG. 1 illustrates that cycler 20 includes one or more pressure sensor 40 placed along actuation surface 22a at patient line 116 to read positive and negative pressure of fresh dialysis fluid delivered to and used dialysis fluid removed from the patient. The pressure readings are likewise used as feedback to control the speed of the peristaltic pump actuator. Any of the feedback loops discussed herein, e.g., temperature or pressure loops, may include a control algorithm such as a proportional, integral and derivative control algorithm. The pressure sensor may be used to ensure that a positive patient pressure limit, for example one to five psig (6.9 to 34.5 kPa, e.g., two psig (13.8 kPa)), or a negative patient pressure limit, e.g., −1.0 psig to −3.0 psig (−6.9 to −20.7 kPa, e.g., −1.3 psig (−9.0 kPa)), is not exceeded during treatment.

Pressure sensor 40 in an embodiment includes a dome having a fluid side and an air side separated by a flexible pressure transmission membrane. Fresh or used dialysis fluid flows through the fluid side of the pressure dome and transmits fluid pressure via flexible pressure transmission membrane to the air side. An air tube runs from the air side to a pressure transducer located within housing 22 of cycler 20. The pressure transducer produces a signal indicative of the positive or negative pressure of the fresh or used dialysis fluid in the patient line.

Pressure sensor 40 in an alternative embodiment includes a force sensor that measures pressure directly at patient line or tube 116 of disposable set 100. Such a pressure or force sensor may be accurately calibrated by moving a defined volume towards, and away from, closed valve 26c. The pressure built depends on the length and material of tube 116. Empirical experiments are performed and one or more table of pressure versus volume is formed and stored at control unit 50 of cycler 20. Control unit 50 may be programmed such that if the pressure built in patient line or tube 116 does not match that of the one or more table, cycler 20 alarms and halts treatment. In such case, perhaps a competitor's line set has been used, there is a leak in disposable set 100, or pressure sensor 40 is not operating properly. It is also contemplated for control unit 50 to take one or more reading from pressure sensor 40 when patient line connector 126 at the distal end of patient line 116 is placed in its priming position in the holder of prime sensor 46. Here, the hydrostatic pressure provided by filled patient line 116 is known knowing the head height of the patient line and the density of the dialysis fluid and can be used as an input for the calibration of pressure sensor 40 assuming the line is properly primed.

FIG. 1 illustrates that cycler 20 may further include at least one pathogen barrier device 44, such as an ultraviolet (“UV”) light source, placed along actuation surface 22a. Pathogen barrier device 44 operating with a drain line 110 of disposable set 100 is intended to prevent pathogens from migrating, e.g., from the toilet, bathtub or sink, up the drain line and into a peristaltic pumping area of disposable set 100. Cycler 20 may further additionally include a prime sensor 46, e.g., an optical, ultrasonic, capacitance, proximity or magnetic sensor, against which the patient line connector is positioned prior to treatment. Prime sensor 46 senses when the patient line has been fully primed so that treatment may begin.

Control unit 50 of cycler 20 in the illustrated embodiment of FIG. 1 includes one or more processor 52, one or more memory 54 and a video controller 56 operating with a user interface 58 to control each of peristaltic pump actuator 24, dialysis fluid valves 26a to 26c, heater 30 and pathogen barrier device 44 or UV light source. Control unit 50 receives signals from each of temperature sensors 32a, 32b, the weigh scale or load cells 34a, 34b, pressure sensor(s) 40 and prime sensor 46. User interface 58 may be provided with a touchscreen and/or electromechanical pushbuttons to allow the user or patient to enter parameters for treatment and a display screen for providing information, such as treatment status information. User interface 58 may include speakers (e.g., operable with a sound card of control unit 50) to output information to the patient or user, e.g., alarms, alerts and/or voice guidance commands. Control unit 50 may also include a transceiver and a wired or wireless connection to a network, e.g., the internet, for sending treatment data to and receiving prescription instructions from a doctor's or clinician's server interfacing with a doctor's or clinician's computer.

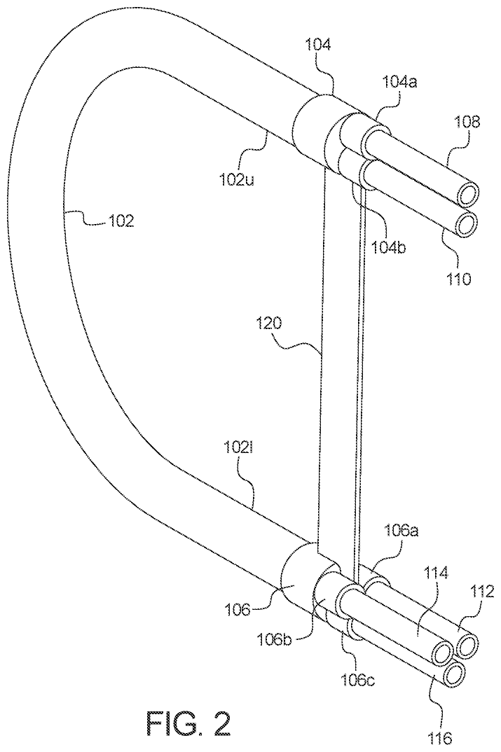

Disposable set 100 as illustrated in FIGS. 1 and 2 includes a peristaltic pump tube 102 that operates with peristaltic pump actuator 24. Peristaltic pump tube 102 is connected at both ends to a disposable connector 104, 106 that splits the single tube 102 into multiple inlet/outlet ports. For example, assuming that peristaltic pump actuator 24 and peristaltic pump tube 102 are mounted vertically, an upper end 102u of peristaltic pump tube 102 connects to upper connector 104, which splits into two inlet/outlet ports, one a heating container port 104a and another a drain line port 104b. A lower end 102ₗ of peristaltic pump tube 102 may connect to a lower connector 106, which splits into three or more inlet/outlet ports, one a patient line port 106a and the other two or more ports being dialysis fluid ports 106b and 106c.

Disposable set 100 in the illustrated embodiment includes a heater line or tube 108 that extends from heating container port 104a to a heating container or bag 118. A drain line 110 extends from drain line port 104b to a house drain, such as a toilet, bathtub or sink. One advantage of the system 10 is that even though it uses a weigh scale 34a, 34b, system 10 does not require a separate drain container, reducing disposable cost and weight. Dialysis fluid lines or tubes 112 and 114 extend from dialysis fluid ports 106a and 106b to dialysis fluid containers or bags 122 and 124, which may hold different dialysis fluid volumes and types, e.g., different glucose or dextrose levels or different formulations. One dialysis fluid container or bag may include icodextrine for a last patient fill. Patient line or tube 116 extends from patient line port 106c to a patient line connector 126, which for example connects to a patient's transfer set leading to the patient's indwelling catheter.

Connectors 104 and 106 that attach to the end of peristaltic pump tube 102 may be made, e.g., molded or extruded, of a rigid or non-rigid polymer. FIG. 2 illustrates that for manufacturing, rigidity and ease of handling, connectors 104 and 106 may be molded together via a bridging member 120. In any case, connectors 104, 106, fluid lines 108 to 116 and fluid containers or bags 118, 124 and 126 of disposable set 100 may be made of one or more plastic, e.g., polyvinylchloride (“PVC”) or a non-PVC material, such as polyethylene (“PE”), polyurethane (“PU”) or polycarbonate (“PC”). Housing 22 of the cycler 20 may be made of any of the above plastics, and/or of metal, e.g., stainless steel, steel and/or aluminum.

In the illustrated embodiment of FIG. 1, first rotating pinch valve valves 26a is operable with heater line or tube 108 and drain line or tube 110, wherein rotating pinch valve valves 26a rotates such that one of the lines or tubes 108, 110 is open, while the other is occluded. Second rotating pinch valve 26b is associated with dialysis fluid lines or tubes 112 and 114, wherein rotating pinch valve valves 26b rotates such that one of the dialysis fluid lines 112 and 114 is open, while the other is occluded. Third rotating pinch valve 26c is associated again with dialysis fluid line or tube 114 and also with the patient line or tube 116, wherein rotating pinch valve valves 26c rotates such that one of dialysis fluid line 114 or patient line or tube 116 is open, while the other is occluded. The combination of second and third rotating pinch valves 26b and 26c allow either of dialysis fluid lines 112 and 114 to be open and patient line 116 to be occluded or for patient line 116 to be open and dialysis fluid lines 112 and 114 to be occluded. The valve arrangement of system 10 allows for fewer valves (e.g., three valves controlling five lines) and thus a more simplified and cost effective cycler 20.

If patient P is initially full of used dialysis fluid, control unit 50 causes peristaltic pump actuator 24 to rotate in a clockwise direction, and with rotating pinch valves 26a to 26c rotated so that patient line 116 and drain line 110 are open, to pull used dialysis fluid from patient P push same to the house drain. The above sequence is repeated for each patient drain. For each patient fill, control unit 50 causes peristaltic pump actuator 24 to rotate in a counterclockwise direction, and with rotating pinch valves 26a to 26c positioned so that heater line 108 and patient line 116 are open, to pull fresh, heated dialysis fluid from heating container or bag 118 and push same to patient P. Heating container or bag 118, may or may not be filled with an initial supply of dialysis fluid. Weigh scale 34a and 34b under control of control unit 50 enables a precise amount of heated, fresh dialysis fluid to be delivered to patient P. To initially fill or replenish heating container or bag 118 with fresh dialysis fluid, control unit 50 causes peristaltic pump actuator 24 to rotate in a clockwise direction, and with rotating pinch valves 26a to 26c operated so that heater line 108 and one of dialysis fluid lines 112 and 114 are open, to pull fresh dialysis fluid from one of dialysis fluid containers or bags 122 or 124 and push same to heating container or bag 118, where the fresh dialysis fluid is heated, e.g., during a patient dwell.

It should be appreciated that with heater line 108 and drain line 110 both located on the same side of peristaltic pump tube 102, there is no way to weigh patient P's effluent and then deliver it to drain. A separate drain container and associated line and valve could be provided, where the dedicated drain container would reside on weigh scale 34a and 34b along with heating container or bag 118, however, such arrangement adds disposable waste, cost and complexity.

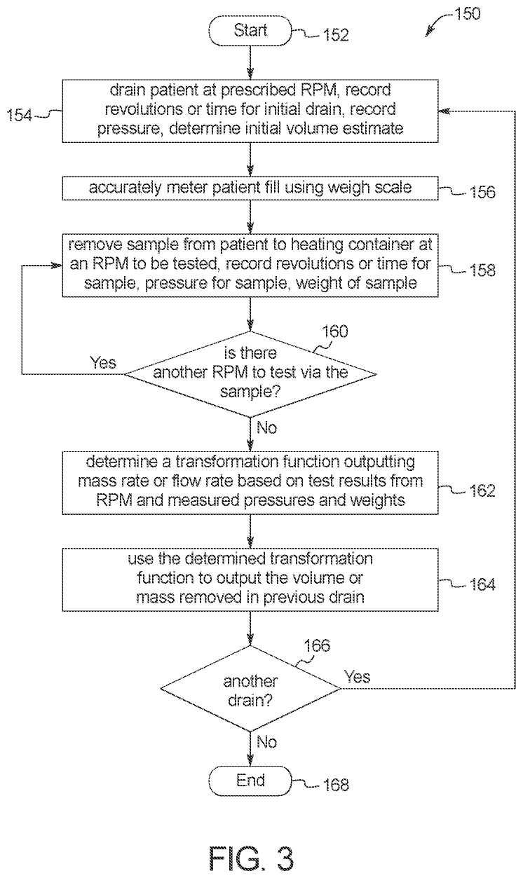

To enable the drain fluid to be measured accurately without the use of weigh scale 34a and 34b, it is contemplated for control unit 50 to run method 150 for system 10 as illustrated in FIG. 3, which may be run at the first fill only, but can also be run after each fill if needed or desired. Method 150 teaches that control unit 50 is programmed to calibrate the peristaltic pump using weigh scale 34a and 34b at one or more flowrate and pressure and then use the calibration for the next one or more drain. At oval 152, method 150 begins. At block 154, assuming that the patient is initially full of used dialysis fluid from a prior treatment (but does not have to be for the calibration of method 150), control unit 50 causes cycler 20 to drain patient P of used dialysis fluid or effluent. Along with the drain, control unit 50 accurately records the number of revolutions (and partial revolution) that peristaltic pump actuator 24 makes during the initial drain and the resulting pressure reading from pressure sensor 40. Control unit 50 may alternatively record the time duration of the initial drain. Control unit 50 commands a prescribed revolutions per minute (“RPM”) to operate peristaltic pump actuator 24 for the initial drain. Control unit 50 also determines the initial drain volume by multiplying the recorded number of peristaltic pump strokes by an expected volume per stroke, which is not accurate, but suffices for the initial drain and for the present time.

At block 156, control unit 50 of cycler 100 causes patient P to be filled with a prescribed amount of fresh, heated fluid, which is measured and metered accurately by weigh scale 34a, 34b. An additional amount of fluid, e.g., around 90 ml, which is a known amount needed to fill patient line 116 from cycler 20 to patient P is also delivered via pump actuator 24 operating peristaltic pump tubing 102.

At block 158, control unit 50 of cycler 100 causes a sample of dialysis fluid (e.g., in an amount equal to the volume of patient line 116) to be removed from patient P to heating container or bag 118, where it is weighed accurately via weigh scale 34a and 34b. The removal is done at one or more revolutions per minute (“RPM”) of the peristaltic pump actuator 24 which is desired to be tested, such as the prescribed RPM for the previous drain, and for a known amount of revolutions and perhaps partial revolution (or over a measured amount of time). A resulting pressure and a weight gain (from which volume may be determined) are measured for the tested RPM.

It may be desirable to test multiple RPM over the withdrawal of the sample. For instance, the drain at block 154 may have been performed over multiple RPM. That is, it is quite possible that a patient drain of system 10 is performed by varying the speed of peristaltic pump actuator 24 over the course of a drain and in particular slowing down peristaltic pump actuator 24 at the beginning and/or end of the drain to improve patient comfort. It is contemplated therefore to test the weight samples at the different speeds that peristaltic pump actuator 24 has been be run over the different stages of the pervious drain. Or it may be desirable to test a range of RPM around a single prescribed RPM used at the previous drain of block 154.

In any case, at diamond 160, if there is another RPM to test, the pressure and weight measurements performed at block 158 are repeated for a different desired RPM. In one example, if a maximum drain rate is an RPM corresponding to 250 ml/min, samples may be taken for RPM expected to yield 100 ml/min, 150 ml/min and 250 ml/min, at least one of which corresponds to the prescribed RPM of the drain performed at block 154 (which may be an initial drain or a subsequent drain). In this manner, the pressure and resulting flowrate for multiple RPM of peristaltic pump actuator 24 are known for the particular peristaltic pump tube in use during the present treatment.

The procedures of block 158 and diamond 160 may be performed in a step-wise manner, where the pump RPM and resulting flowrates are held constant for a period of time or revolutions so that the corresponding load cell and pressure readings may steady or equalize before control unit 50 advances to the next RPM. Alternatively, the procedures of block 158 and diamond 160 may be performed continuously or virtually continuously by changing over a very small amount of time or partial revolution (e.g., input current or input pulse frequency to peristaltic pump actuator 24 follows a constantly rising slope), starting from zero and rising to a maximum (e.g., set maximum or a maximum flowrate seen during an initial/previous patient drain). Control unit 50 here measures weight changes via load cells 34a, 34b and the pressure changes via pressure sensor 40 continuously and correspondingly.

At block 162, control unit 50 determines a transformation function or algorithm that outputs flowrate or mass rate (and hence volume or mass) as a function of pressure and RPM (e.g., Q=f(RPM; P)). That is, from the testing of the sample, control unit 50 correlates each tested RPM with its measured pressure and resulting weight change. The transformation function may be in the form of a lookup table that outputs flowrate or mass rate from an inputted RPM and pressure. The transformation function may alternatively be in the form of an algorithm that calculates flowrate or mass rate from an inputted RPM and pressure.

At block 164, control unit 50 applies the transformation function determined at block 162 to the RPM and pressures of the drain at block 154 (which may be an initial or subsequent drain), allowing control unit 50 to determine and record an accurate mass rate and/or volume rate for the initial drain. The mass rate and volume rate is on one embodiment based on pump actuator strokes. In the case of peristaltic pump actuator 24, the pump stroke is a pump revolution (or partial revolution). The totaled revolutions (and possible partial revolution at the end) for the drain at block 154 multiplied by one or more mass rate or volume rate corresponding to the one or more RPM and resulting pressure used during the drain at block 154 yields an accurate total weight or volume of the drain. At block 164, control unit 50 accordingly replaces the previously calculated (not accurate) volume of the drain at block 154 with the more accurate follow-up total weight or volume of the drain just described.

It is known that peristaltic pump tubing 102 wears over the course of treatment, so the resulting mass rate or flowrate and pressure for a given RPM may be different during a subsequent drain versus a previous drain. At diamond 166, control unit 50 determines whether there is another drain, and if so, returns to block 154 for the subsequent drain upon which method steps 154 to 164 just described are repeated. If there is no subsequent drain as determined at diamond 166, then method 150 ends at oval 168. Control unit 50 may follow method 150 with a last fill of fresh, heated dialysis fluid, e.g., icodextrine, which remains with the patient until a next treatment or day exchange.

In an alternative embodiment, control unit 50 at method 150 does not have to take a new sample and determine a new transformation function for each drain. Instead, control unit 50 may use a determined transformation function for multiple drains or perhaps each drain of a treatment. It may be determined that peristaltic tubing wear does not affect performance significantly over a certain number of drains, say at the beginning of treatment. In such case, the sampling and transformation function determination may be skipped for one or more drain phase. It should be appreciated however that the application of a transformation function at block 164 is still performed albeit with a prior determined transformation function.

Again, it may be determined that the amount of wear over multiple drains does not significantly affect the performance of the peristaltic pump. If so, it may be possible for control unit 50 to determine an accurate RPM at which peristaltic pump actuator 24 is to be operated so as to achieve a prescribed flowrate. For example, suppose the RPM expected to yield 100 ml/min, 150 ml/min and 250 ml/min for certain pressure(s) are instead found via the sampling to instead yield 98 ml/min, 147 ml/min and 245 ml/min, respectively. Here, control unit 50 may be programmed to notice that the actual flowrates are consistently or on average two percent less than the expected flowrates. Control unit 50 may thereafter increase one or more RPM by two percent in an attempt to generate a prescribed flowrate. In a subsequent sample, control unit 50 may test the increased one or more RPM, which is weighed and measured accurately to confirm that the prescribed flowrate is met and that the resulting negative pressure is at or within a negative patient pressure limit, e.g., −1.0 psig to −3.0 psig (−6.9 to −20.7 kPa, e.g., −1.3 psig (−9.0 kPa)). If confirmed, the next patient drain can be operated with peristaltic pump actuator 24 running at the increased one or more RPM, and wherein the negative pressure is monitored to confirm it matches the pressure recorded during the sample.

The calibration procedure of method 150 of system 10 enables the drain amounts to be recorded accurately without having to collect the patient's effluent in a separate drain container for weighing, which would have to be swapped out or loaded in combination with heating container or bag 118 to use with weigh scale load cells 34a, 34b. The patient fill amounts are measured and metered via weigh scale load cells 34a, 34b. At the end of treatment, the total amount of used dialysis fluid removed from the patient (e.g., including an initial removal from the previous day's last fill) less the total amount of fresh dialysis fluid delivered to the patient (including present day's last fill amount), as determined by control unit 50, yields the overall amount of ultrafiltration (“UF”) removed from the patient over the treatment period.

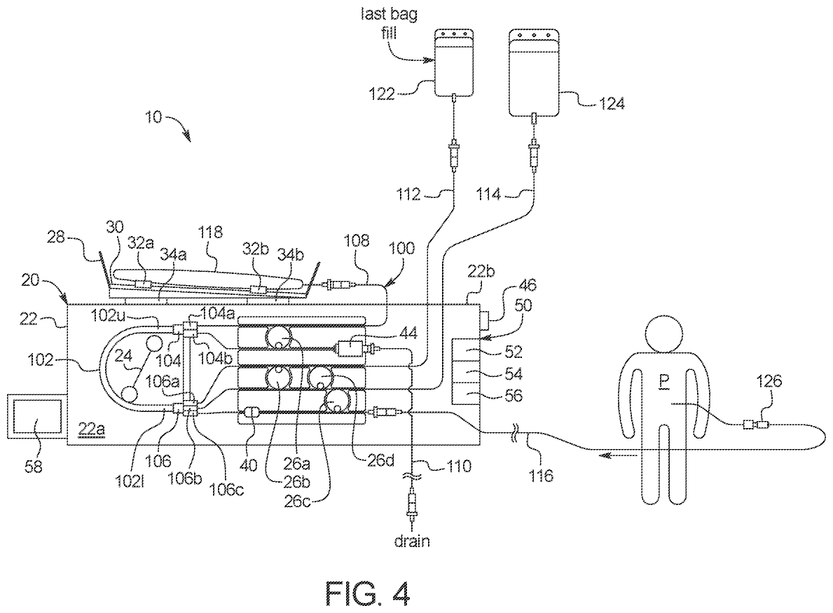

FIG. 4 illustrates an alternative embodiment for system 10. System 10 is advantageous because it allows patient drain amounts to be determined accurately without having to weigh the patient's effluent, which would require either a separate drain container located above heating container or bag 118, increasing disposable cost, or delivering the patient effluent to heating container or bag 118, where it may not be desirable to pump fresh PD fluid and patient effluent to the same location. In FIG. 1, the sample from the patient fill allows peristaltic pump actuator 24 to rotate in a patient drain direction (e.g., clockwise) and to deliver the sample to fluid heating container or bag 118 for weighing. In the alternative embodiment of FIG. 4, fresh PD fluid delivered from fluid containers or bags 122 and 124 to replenish heating container or bag 118 is used instead of (or in addition to) the sample. Here again, peristaltic pump actuator 24 rotates in the patient drain (e.g., clockwise) direction.

Method 150 of FIG. 3 for the alternative embodiment of FIG. 4 is performed by control unit 50 in the same way as described above, except blocks 156 and 158 for FIG. 4 occur at the same time, wherein block 156 instead involves replenishing heating container or bag 118 from fluid containers or bags 122 and 124, and wherein block 158 instead involves (i) replenishing heating container or bag 118 at an RPM to be tested, (ii) recording revolutions or time for the tested RPM, (iii) recording pressure for the tested RPM, and (iv) detecting a change of weight for the tested RPM. Diamond 160 instead inquires whether there is another RPM to test during the replenishing of heating container or bag 118. Here, it should be appreciated that a longer time to test different RPM is provided in the alternative embodiment of FIG. 4 (replenishing may be on the order of two liters, while the sample for FIG. 1 may be around 90 ml). Blocks 162 and 164 and diamond 166 remain unchanged for the alternative embodiment of FIG. 4.

During the replenishing of heating container or bag 118 from fluid containers or bags 122 and 124, control unit 50 causes patient line 116 to be occluded via rotating pinch valve 26_{c} and drain line 110 to be occluded via rotating pinch valve 26ₐ. If replenishing is performed from fluid container or bag 124 via line 114, then control unit 50 causes rotating pinch valve 26_{b} to occlude dialysis fluid line or tube 112. If replenishing is performed instead from last fill container or bag 122 via line 112, then control unit 50 causes rotating pinch valve 26_{b} to occlude dialysis fluid line or tube 114. A fourth rotating pinch valve 26d under control of control unit 50 may be provided to variably, but not completely, occlude the open dialysis fluid line or tube 112 or 114 to achieve a desired pressure (negative) to the inlet of peristaltic pump tube 102. In this manner, control unit 50 may ensure that desired pressures, e.g., pressures that have occurred during a prior drain, are interrogated during the replenishing sequence. If it is found that pressure control via the speed of peristaltic pump actuator 24 alone is sufficient to interrogate the needed prior drain pressures, then fourth rotating pinch valve 26d may not be needed.

It is contemplated that with patient line 116 occluded in FIG. 4, pressure sensor 40 can detect the negative pressure introduced through either dialysis fluid line or tube 112 or 114 via the common fluid pathway at lower end 102ₗ of peristaltic pump tube 102, which is in fluid communication with all three lines 112, 114 and 116. If not, it is contemplated to position and arrange additional pressure sensors 40 so as to operate with dialysis fluid lines or tubes 112 or 114. Here, control unit 50 would selectively operate with the relevant pressure sensor (for line 112 or 114) during the replenishing, while the patient line pressure sensor 40 would be used primarily for pumping pressure control.

While the embodiment of FIG. 4 has been described as an alternative, the embodiment of FIG. 4 may be provided and used in addition to the embodiment of FIG. 1, where the results of each are combined, e.g., averaged, etc. The embodiment of FIG. 1 occurs directly after a fill of patient P, while the embodiment of FIG. 4 likely occurs during a subsequent patient dwell, so there is time to perform both embodiments if desired. It should also be appreciated that there is an initial filling of heating container or bag 118, which may occur prior to an initial patient drain to allow the first patient fill PD fluid to begin heating as soon as possible. Thus it is contemplated to use the transformation function results from the first or initial filling of heating container or bag 118 for a subsequent initial drain of patient P and to continue to use predetermined transformation function results for subsequent drains over the course of treatment. One drawback here is that the RPM and pressures to be interrogated are not already known from a prior drain and instead are assumed from historical drain RPM and pressures.

It should be understood that various additional changes and modifications to the presently preferred embodiments described herein will be apparent to those skilled in the art. It is therefore intended that such changes and modifications be covered by the appended claims. For example, cassette-based valves, such as volcano valves, or linearly actuated pinch valves, may be used instead of the above-described rotary pinch valves. In another example, any type of pump that may benefit from use with a weigh scale may be used, e.g., a gear pump, membrane pump or centrifugal pump. Such pumps may have different types of strokes and stroke rates (e.g., non-rotational) than peristaltic pump actuator 24 discussed herein, however, the mass rate or volume rate per stroke may still be determined. Also, while the testing is described herein being performed with dialysis fluid, the testing may be performed with any suitable, e.g., sterile fluid. For example, if system 10 is an online system that forms dialysis fluid with purified water and concentrate, either the water or concentrate may be delivered to patient line 116 and then pulled back into heating container or bag 118 at which time the testing is performed and the matrix is determined. The water or concentrate is then mixed to form dialysis fluid.

The invention is claimed as follows:

1. A peritoneal dialysis system comprising:
a cycler including
a peristaltic pump actuator, and
a weigh scale;
a disposable set including
a peristaltic pump tube operable with the peristaltic pump actuator, and
a heating container in operable communication with the weigh scale; and
a control unit configured to (i) cause the peristaltic pump actuator to operate at a rotational speed to deliver an amount of fluid to the heating container, (ii) record a weight of the amount of fluid from the weigh scale, and (iii) determine at least one of a mass rate or volume rate for the rotational speed using the recorded weight.

2. The peritoneal dialysis system of claim 1, wherein at least one of (i) the fluid is dialysis fluid, purified water or concentrate, or (ii) the control unit is provided with the cycler.

3. The peritoneal dialysis system of claim 1, wherein the control unit is further configured to use the at least one of the mass rate or volume rate for the rotational speed to determine a mass or volume of used dialysis fluid removed in a prior drain operated at the rotational speed.

4. The peritoneal dialysis system of claim 1, wherein the control unit is configured to perform (i) to (iii) after each of a plurality of patient drains.

5. The peritoneal dialysis system of claim 1, wherein the amount of fluid entering the heating container during (i) is supplied as a sample taken from dialysis fluid provided during a patient fill to the patient.

6. The peritoneal dialysis system of claim 1, wherein the amount of fluid entering the heating container during (i) is supplied via a heating container replenishing sequence.

7. The peritoneal dialysis system of claim 1, wherein the cycler further includes a pressure sensor, wherein the control unit records at least one reading from the pressure sensor during (i), the at least one reading associated with the rotational speed.

8. The peritoneal dialysis system of claim 7, wherein the control unit is further configured to monitor at least one reading from the pressure sensor during a prior drain while actuating the peristaltic pump tube at the rotational speed.

9. The peritoneal dialysis system of claim 7, wherein the control unit is configured to determine a transformation function that relates flowrate or mass rate as a function of the rotational speed and the at least one reading from the pressure sensor.

10. The peritoneal dialysis system of claim 9, wherein the transformation function is provided as a lookup table or an algorithm.

11. The peritoneal dialysis system of claim 9, wherein the control unit is configured to apply the transformation function to at least one rotational speed used during a prior drain.

12. The peritoneal dialysis system of claim 1, wherein the control unit is configured to repeat (i) to (iii) at least one time at different rotational speeds.

13. The peritoneal dialysis system of claim 12, wherein the control unit is configured to repeat (i) to (iii) multiple times starting from a first rotational speed and increasing incrementally to a maximum rotational speed.

14. The peritoneal dialysis system of claim 12, wherein the control unit is configured to use (i) the rotational speed and the at least one different rotational speed occurring during a prior patient drain and (ii) the corresponding mass rate or volume rate for the rotational speed and the at least one different rotational speed to determine the weight or volume of used dialysis fluid removed over the prior patient drain.

15. The peritoneal dialysis system of claim 12, wherein the control unit is configured to use the mass rate or volume rate for the rotational speed and the at least one different rotational speed to determine at least one adjusted rotational speed to use for a subsequent patient drain to achieve a prescribed drain volume.

16. A peritoneal dialysis system comprising:
a cycler including
a peristaltic pump actuator,
a weigh scale, and
a pressure sensor,
a disposable set including
a peristaltic pump tube operable with the peristaltic pump actuator,
a patient line in fluid communication with the peristaltic pump tube, wherein the pressure sensor is positioned and arranged to measure pressure in the patient line, and
a heating container in operable communication with the weigh scale; and
a control unit configured to (i) cause the peristaltic pump actuator to operate at a rotational speed to deliver an amount of fluid to the heating container, (ii) record a weight of the amount of fluid from the weigh scale, (iii) record a pressure of the fluid from the pressure sensor, and (iv) determine a transformation function that relates flowrate or mass rate as a function of the rotational speed and the recorded pressure.

17. The peritoneal dialysis system of claim 16, wherein the control unit is configured to apply the transformation function to at least one rotational speed used during a prior drain.

18. A peritoneal dialysis system comprising:
a cycler including
a peristaltic pump actuator, and
a weigh scale;
a disposable set including
a peristaltic pump tube operable with the peristaltic pump actuator, and
a heating container in operable communication with the weigh scale; and
a control unit configured to (i) cause the pump actuator to actuate the pumping portion at a strokes per minute (“SPM”) to deliver an amount of fluid to the heating container, (ii) record a weight of the amount of fluid from the weigh scale, and (iii) determine at least one of a mass rate or volume rate for the SPM using the recorded weight.

19. The peritoneal dialysis system of claim 18, wherein the strokes depend on rotational movement, piston movement, membrane movement or centrifugal movement.

20. The peritoneal dialysis system of claim 18, wherein the control unit is configured to repeat (i) to (iii) at least one time at a different SPM.

21. The peritoneal dialysis system of claim 20, wherein the control unit is configured to repeat (i) to (iii) multiple times starting from a first SPM and increasing incrementally to a maximum SPM.

22. The peritoneal dialysis system of claim 20, wherein the control unit is configured to use (i) the SPM and the at least one different SPM occurring during a prior patient drain and (ii) the corresponding mass rate or volume rate for the SPM and the at least one different SPM to determine the weight or volume of used dialysis fluid removed over the prior patient drain.

23. The peritoneal dialysis system of claim 20, wherein the control unit is configured to use the mass rate or volume rate for the SPM and the at least one different SPM to determine at least one adjusted SPM to use for a subsequent patient drain to achieve a prescribed drain volume.

24. The peritoneal dialysis system of claim 18, wherein the cycler further includes a pressure sensor, and wherein the control unit records at least one reading from the pressure sensor during (i), the at least one reading associated with the SPM.

25. The peritoneal dialysis system of claim 24, wherein the control unit is configured to determine a transformation function that relates flowrate or mass rate as a function of the SPM and the at least one reading from the pressure sensor.

26. The peritoneal dialysis system of claim 25, wherein the control unit is configured to apply the transformation function to at least one SPM used during a prior drain.
